(19) United States
(12) Patent Application Publication
(10) Pub. No.: US 2006/0251605 A1
(43) Pub. Date: Nov. 9, 2006

(54) Method to prepare personal care composition from a concentrate

(76) Inventors: Maria Teresa Belmar, Shamrock (GB); Jason Stokes, Shamrock (GB); Julia Telford, Shamrock (GB); Shiping Zhu, Shamrock (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/547,915

(22) PCT Filed: Feb. 20, 2004
(86) PCT No.: PCT/EP04/01753

§ 371 Date: Jul. 3, 2006

(30) Foreign Application Priority Data
Mar. 12, 2003 (EP) 03251495.2

(51) Int. Cl. A61K 8/41; A61K 8/37; A61K 8/36

(52) U.S. Cl. 424/70.22; 424/70.31

(57) ABSTRACT

A process is provided for preparing a personal care composition, which is based on combining a concentrate comprising neutralising base and surfactant with water and sensory ingredients, functional ingredients or a combination thereof.

### FIELD OF THE INVENTION

The invention relates to a process for preparing a personal care product such as a cream or lotion wherein the process enables the production of a large variety of products in an efficient way.

### BACKGROUND

Personal care compositions such as skin creams and lotions are typically prepared on an industrial scale in a centralised manner, using large-scale equipment. In such a typical production process, for the production of products which are typically emulsions, the highest melting point ingredients are typically heated in a large container until they are molten (typically at around 80° C.), stirred, and further ingredients are added to the composition as it is continually stirred and cooled. The last of these ingredients is often perfume, which may be added when the composition is typically at around 40° C. Thereafter, the composition is cooled and dosed into retail containers. The time and speed of shearing used in the process typically effects the droplet size of the resulting emulsion.

A number of problems may be associated with the common large-scale methods of manufacture. Foremost of these is the difficulty of individualization or customization of products, with the result that typically only relatively few products may be provided to the consumer. Preparation of customized products by changing a minor feature e.g. color, fragrance, presence/amount of promotionals or minor components such as vitamins and retinol is however a desire that exists among users of skin creams and lotions.

A disadvantage of the known production process for producing skin creams and lotions is the time and cost associated with changing products on a production line; because of the need to thoroughly clean the production apparatus between production runs of different products, especially with large scale manufacturing equipment, this contributes significantly to the difficulties of producing many different products on such a line within a reasonable time scale.

As a consequence of this, where a product is a customized or individualized product, and the batch of that particular product is relatively small, the cost of the product is relatively high.

In addition, a typical skin cream composition may easily comprise 60-90% water. As such, water makes up a large proportion of the weight of a topical product. It therefore represents a large proportion of the cost of transporting such a commercial product between manufacture and retailing. Further more, the cost for heating and cooling the water in the preparation is also high and the processing time is usually long, 3-5 hrs.

As a consequence of the complicated known production process only a limited variety of products are made by industrial producers of personal care products which rely on central manufacturing of their products.

Even if there is variety, it is often only in certain aspects e.g. color, fragrance and minor ingredients because these may be post-added to a finished base product.

Usually texture of the cream, feel on skin, moisturizing properties can not be varied.

It is an object of the current invention to overcome this drawback of central manufacturing of personal care products.

### SUMMARY OF THE INVENTION

We have now found that it is possible to provide a wide variety of products when starting from a specific aqueous concentrate and treating that in a few carefully selected steps.

Therefore the invention relates to a process for the preparation of a personal care composition, which process comprises the steps of
a) Providing an aqueous concentrate composition comprising a neutralising base and 6 to 90 wt % of a surfactant at a temperature of from 60 to 150° C., preferably from 70 to 90° C.; said surfactant comprising a fatty acid
b) Cooling the composition obtained in step (a) to a temperature below 60° C., preferably to a temperature from 5 to 30° C., more preferred from 10 to 25° C. whereby below a temperature of 65° C., the cooling is preferably carried out quiescently
c) dilution of the base composition until a surfactant concentration between 1 and 30% is obtained
d) setting the sensory perception and the functionality of the composition by adding at least one ingredient selected from the group comprising sensory ingredients, functional ingredients or a combination thereof,
e) Optionally adding promotionals and/or emotionals or other minor ingredients;
   wherein steps c, d, and e may be performed simultaneously or sequentially in any order.

The preferred order of steps is first (c), then (d) and (e).

### DETAILED DESCRIPTION

The invention provides a method for preparing a personal care composition, preferably a cosmetic composition such as a skin cream. Such compositions include but are not limited to skin creams, lotions, vanishing creams, shampoos and the like. Preferably the composition can be easily spread on the skin.

A preferred composition is characterized by an apparent viscosity of from 0.1 to 10000 Pa·s at a shear rate of 10 s⁻¹ to 100 s⁻¹, preferably from 0.1 to 100 Pa·s, more preferred from 0.1 to 20 Pa·s at a shear rate of 10 s⁻¹ or an apparent yield stress of from 0.1 to 10000 Pa, preferably from 1 to 10000 Pa, more preferred from 5 to 1000 Pa, even more preferred from 10 to 100 Pa.

In the context of the invention, a crystallization temperature or phase transition temperature may be determined by using DSC techniques.

In the process for the preparation of a personal care composition, in a first step (a) an aqueous concentrate composition comprising a neutralizing base and 6 to 90 wt %, preferably 10 to 90 wt %, of a surfactant is provided at a temperature of from 60 to 150° C. The surfactant comprises fatty acid.

This aqueous composition may be prepared in any suitable way e.g. by mixing surfactant with water and base material and subsequently heating this mixture to a temperature from 60 to 150° C. Alternatively (part) of the ingredients of this concentrate composition are pre-heated to the desired temperature before setting the temperature of the mixture to the desired temperature of between 70 and 90° C.

It is preferred that the aqueous concentrate composition is mixed such that a homogeneously mixed composition is provided in step (a).

It is highly preferred that the aqueous composition is prepared as such from its ingredients and remains in liquid form. However, in an alternative method it is possible to form the aqueous concentrate and then subject it to drying. The resulting anhydrous composition may be re-constituted to form an aqueous concentrate for further handling in step (c).

The temperature of the composition provided in step (a) is from 60 to 150° C. Within this range the temperature is preferably chosen such that it is above the crystallization temperature of the mixture provided. Generally a suitable temperature is from 70 to 90° C.

The composition in step (a) may also be referred to as concentrate. The concentrate preferably comprises a fatty acid as surfactant, a neutralizing base and at least one other surfactant component selected from the group comprising long chain fatty alcohol, fatty acid esters, polyethylene glycol, glycerol monostearate, cetylalcohol, or a combination thereof. Other suitable surfactants are polyethylene oxide esters.

The neutralising base in the concentrate is preferably selected from the group comprising organic base, such as triethanolamide, diethanolamine, monoethanolamide, triisopropanalamine, inorganic base such as KOH, or a mixture of inorganic and organic base, and/or a non ionic surfactant. Most preferred the neutralising base is an organic base, which optionally comprises a small amount (up to 30 wt % on total base) of an inorganic base.

It is preferred that the neutralising base composition, especially organic base, is added in the form of an aqueous composition. This ensures maximum structure formation. Alternatively the organic base is e.g. added with the surfactants.

In a preferred embodiment, the amount of neutralising base in step (a) is such that the fatty acid is partially neutralised. This means that only part of the fatty acids together with the neutralising base forms a soap and that there is also free fatty acid left. By controlling the ratio of the fatty acid materials to base, it is possible to control the degree of saponification of the fatty acid materials. This saponification is also referred to as neutralisation. Preferably the degree of neutralisation is 8 to 90% for the fatty acids present in the surfactant.

Preferred fatty acids are characterised by a hydrocarbon chain length of from 14 to 20 carbon atoms, more preferred from 16 to 20 carbon atoms, most preferred from 16 to 18 carbon atoms.

In a further step (b) the composition obtained in step (a) is cooled to a temperature below 60° C., preferably to a temperature from 5 to 30° C., more preferred from 10 to 25° C. Without wishing to be bound by any theory it is believed that this cooling serves to obtain crystallization of the surfactants in the base composition. It was surprisingly found that the cooling is preferably carried out under conditions whereby shear is avoided especially when the temperature is brought below the crystallization temperature of (any of) the ingredients present in the base composition. If cooling is not done under such conditions, satisfying products may be obtained but the resulting products will not have optimal structure properties as evidenced by a maximum yield stress. Therefore in a preferred embodiment, the invention relates to a process whereby below a temperature of 65° C., preferably below 60° C., the cooling is carried out quiescently.

In the context of the invention quiescent is defined as without substantial shear. This implies that some shear (e.g. due to movement of the container or tube in which the cooling is carried out, or by stirring in a vessel at a rate per minute below 1,) is tolerable. Preferably the cooling is carried out quiescent over the whole temperature range over which the composition is cooled. It is preferred that quiescent cooling is at least applied below a temperature of 60° C. to avoid interference of shear during crystallization of (part of) the ingredients of the concentrate composition.

The cooling is preferably carried out at a rate of from 0.1 to 20° C. per minute, preferably from 0.5 to 3° C./minute.

Optionally the concentrate in step (a) is provided with other ingredients such as waxes (e.g. petrolatum). It is preferred to include high melting ingredients in step (a) where the temperature is still relatively high because generally such ingredients may not distribute homogeneously once temperatures are low, e.g below 30° C., after step (b).

In a further step (c) the base composition is diluted until a surfactant concentration between 1 and 30 wt %, preferably 2.5 and 30 wt %, more preferably 2.5 and 12 wt % on total product weight is obtained. The total product weight is the weight of the final personal care product.

It was found that the selection of the final surfactant concentration by choosing a suitable degree of dilution enables the production of products with a varying degree of texture/structure. Generally the higher the degree of dilution, the softer, more flowable the products will be. It will be appreciated that the dilution step is an essential step in the process of the invention. Hence in an exemplary embodiment, when the starting concentrate is at a surfactant concentration of 12-30 wt %, the dilution is such that the concentration of surfactant in the final personal care product is lower than that in the concentrate, preferably between 2.5 and 12 wt %. In a preferred embodiment, the personal care composition is a cream or a lotion and the dilution in step (c) is to from 1.5 to 30 wt % surfactant.

The dilution in step (c) may be carried out in any suitable apparatus. It is preferred that minimum shear is used to obtain a homogeneous dilution product.

Mixing conditions comparable to manual mixing of 100 ml of composition with a spatula were found to be suitable.

The dilution in step (c) is preferably with an aqueous composition. Most preferred the aqueous composition that is used for dilution consists essentially of water.

Generally it is preferred that the temperature during the process in step (c), (d) and (e) is from 5 to 90° C., preferably 10 to 90° C., more preferably from 10 to 40° C., most preferred 20 to 40° C.

The dilution in step (c) is preferably carried out at a temperature of the concentrate of from 5 to 95° C. and a temperature of the added aqueous phase for dilution at from 5 to 95° C. It is preferred that the temperature of the added aqueous phase and the concentrate in step (c) are both below 60° C., preferably from 10 to 40° C., more preferred 15 to 40° C., most preferred around room temperature.

One of the main advantages of the present process is that it allows the use of one unique aqueous concentrate composition for the preparation of a large variety of products that may differ in sensory properties. This wide variation is obtained by on the one hand the dilution step which results in a variety of textures, depending on the dilution factor that is chosen, and on the other hand on the step wise addition of sensory/functional ingredients and promotionals/emotionals and other ingredients.

To obtain the desired variation for customization of products in step (d) the sensory perception and the functionality of the composition are set by adding an ingredient selected from the group comprising sensory ingredients, functional ingredients or a combination thereof.

Functionality of the composition, in the context of the invention, is defined as having an effect on moisturisation degree, sun protection factor, water resistance or a combination thereof. The primary functionality of personal care products according to the invention is generally in their moisturisation capacity. Other functionalities are anti-ageing and effect on firmness.

Generally the functionality of a personal care composition may be controlled by the level and variation of emollients and humectants.

Examples of humectants are glycerol(glycerine), oils, and other alcohols. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. The amount of humectant may range anywhere from 0.5 to 30%, preferably between 1 and 15% by weight of the final personal care composition.

Emollients are typically oil based and in liquid form and may be selected from the group comprising hydrocarbons, silicones, synthetic esters or vegetable esters or their combinations.

Silicone oils may be divided into the volatile and non-volatile variety. The term “volatile” as used herein refers to those materials, which have a measurable vapour pressure at ambient temperature. Volatile silicone oils are preferably chosen from cyclic or linear polydimethyl siloxanes containing from 3 to 9, preferably from 4 to 5 silicon atoms. Linear volatile silicone materials generally have viscosities less than about 5 centistokes at 25° C., whilst cyclic materials typically have viscosities of less than about 10 centistokes.

Non-volatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile silicone emollients useful in the present compositions are the polydimethyl siloxanes having viscosities from about 10 to about 400 centistokes at 25° C.

Among the esters are:

(1)Alkenyl or alkyl esters of fatty acids having 10 to 20 carbon atoms. Examples thereof include isoarachidyl neopentanoate, isononyl isononanoate, oleyl myristate, oleyl stearate, and oleyl oleate.
(2)Ether-esters such as fatty acid esters of ethoxylated fatty alcohols.
(3)Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4)Wax esters such as beeswax, spermaceti, myristyl myristate, stearyl stearate and arachidyl behenate.
(5)Sterol esters of which cholesterol fatty acid esters are examples thereof.

Preferred emollients are selected from the group comprising isoparraffins, mineral oil, cyclic or linear polydimethylsiloxanes, vegetable based oils such as sunflower oil and olive oil or a combination of any of these.

Sensory properties relate to sensorial attributes of the product perceived by the user and may include: smoothness, roughness, ease of rub-in, greasiness, thickness, dragginess, silkiness, velvety, stickiness. Such properties may be controlled via addition of ingredients known to give a sensorial response. In particular, the inclusion of a polymeric ingredients that have been specifically found to control the perception of silkiness include Simugel and Sepigel, and modified starches. Silicon oils are found to give a smooth feeling product. High levels of glycerol and thickeners such as Carbopol give ‘sticky’ products. High levels of vegetable oils tend to give a greasy product. The level and ingredient type may be varied to give a desired or satisfactorial sensorial response based on its functionality to satisfy the customer requirements. Thickeners and/or viscosifiers are optionally included to alter the consistency and thickness of the composition and those used include biopolymers, polymers, starches, clays and combinations. The silking agents such as Simugel, sepigel, and starches, may also be used to thicken the composition.

Any combination of the above ingredients may be used to obtain the desired list of sensorical properties.

While most ingredients specifically influence sensorical properties or functional properties, some of the known ingredients will influence both the functional and sensorical characteristics of a composition. The inclusion of such ingredients is preferred.

Therefore in a preferred embodiment, in step (d) the functional and sensorical properties of the composition are set by inclusion of ingredients preferably selected from the group comprising oils (vegetable oils, silicon oils), polymers, and glycerol or a combination thereof.

In a most preferred embodiment, in step (d) the polymers are selected from the group comprising sepigel™, simugel™, polyacrylamide, polyacrylates, silicon polymers, siloxane, modified starches or a combination thereof.

If the process includes a step wherein oil and glycerol are added to the composition, these are preferably added separately, one after the other without a preference for a specific order. They may also be added simultaneously.

In one embodiment, the functional ingredients in step (d) may beneficially comprise little additional thickener. Suitable thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Alternative thickener is clay. Amounts of the thickener may range from 0.0001 to 2%, usually from 0.001 to 1%, by weight of the personal care composition, if at all.

Optionally, in a further step (e) promotionals, emotionals, actives, or other minor ingredients may be added.

Actives are defined as skin benefit agents other than emollients and other than ingredients that merely improve the physical characteristics of the composition. Although not limited to this category, general examples include additional anti-sebum ingredients such as talcs and silicas, and sunscreens. Further examples include silk protein, fragrances, colouring agents, healthy skin ingredients such as AHA, collagen, amino acids; vitamins such as vitamin A and vitamin E, triple lipids such as lecithin, soy sterol; or combinations thereof.

Another minor ingredient for optional addition in step (e) according to the invention may be a retinoid. Retinoids increase collagen synthesis by dermal fibroblasts. This results in protection from sun damage and smoothening of wrinkled skin. The term “retinoids” as used herein, includes retinoic acid, retinol, retinal, and retinyl esters. Included in the term “retinoic acid” are 13-cis retinoic acid and all-trans retinoic acid.

The term “retinol” as used herein includes the following isomers of retinol: all-trans-retinol, 13-cis-retinol, 11-cis-retinol, 9-cis-retinol, 3,4-didehydro-retinol. Preferred isomers are all-trans-retinol, 13-cis-retinol, 3,4-didehydro-retinol, 9-cis-retinol. Most preferred is all-trans-retinol, due to its wide commercial activity.

Retinyl ester is an ester of retinol. The term “retinol” has been defined above. Retinyl esters suitable for use in the present invention are C₁-C₃₀ esters of retinol, preferably C₂-C₂₀ esters, and most preferably C₂, C₃, and C₁₆ esters because they are more commonly available. Examples of retinyl esters include but are not limited to: retinyl palmitate, retinyl formate, retinyl acetate, retinyl propionate, retinyl butyrate, retinyl valerate, retinyl isovalerate, retinyl hexanoate, retinyl heptanoate, retinyl octanoate, retinyl nonanoate, retinyl decanoate, retinyl undecandate, retinyl laurate, retinyl tridecanoate, retinyl myristate, retinyl pentadecanoate, retinyl heptadecanoate, retinyl stearate, retinyl isosterate, retinyl nonadecanoate, retinyl arachidonate, retinyl behenate, retinyl linoleate, retinyl oleate, retinyl lactate, retinyl glycolate, retinyl hydroxy caprylate, retinyl hydroxy laurate, retinyl tartrate.

If present, the retinoids in the present invention may be present in an amount of from 0.001% to 10%, preferably from 0.01% to 1%, and most preferably from 0.01% to 0.05% by weight of the final personal care composition.

For all above identified ingredients that may be added in step (d) or (e), it is optional that the ingredients are present in the form of pre-mixes before they are added. For example minor ingredients may be pre-mixed with water before they are included, to facilitate their dosing.

Alternatively the above-identified ingredients are added as such or mixed with other ingredients before addition.

In a preferred embodiment of the process according to the invention the aqueous concentrate composition is a flowable composition. Also a pumpable paste may suitably be used as a concentrate for step (a). It is preferred that all further ingredients that are added are flowable/pumpable as well rather than being powders that need special handling. In the event that the use of powders may not be avoided, they are preferably added as dispersion in water or oil.

The process according to the invention may be carried out in a central manufacture set up such as a factory line. The process is suitable for application on a large, industrial scale because it starts from a suitable concentrate composition, followed by processing and addition of ingredients such that individual variants.

According to an alternative embodiment the concentrate and the process are carried out at smaller scale in an apparatus made suitable therefore. Such apparatus may be placed in a shop, a distribution centre, small factory outlet or other retail outlet and would enable the consumer to select the specific variety needed. From a concentrate the final product is prepared by use of the process according to the invention.

In an even further embodiment, the concentrate may be sold as such and used to prepare individual products at small scale. In this embodiment, an individual consumer may use the concentrate to prepare her individual personal care product at home in the amount desired and with added ingredient according to her own needs.

In a further aspect the invention relates to use of a process according to the invention to provide a wide variety of personal care compositions which vary in sensorial and functional characteristics.

The process according to the invention is also suitable for use in controlling the sensorial and functional properties of a personal care composition.

### EXAMPLES

General: Determination of Yield Stress

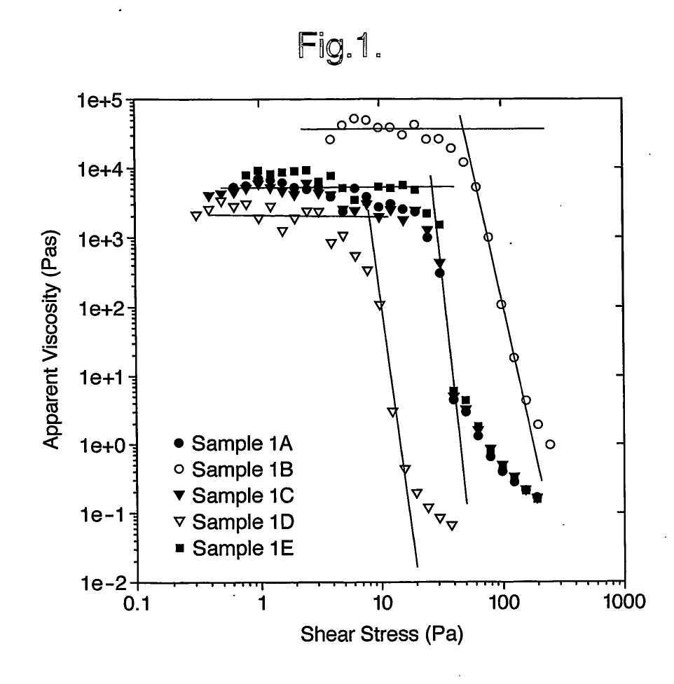

FIG. 1 shows the rheological flow curves for skin cream samples 1A to 1E. The apparent yield stress is specified as the intersection of the lines representing the zero-shear viscosity plateau and the steep decrease is viscosity as shown.

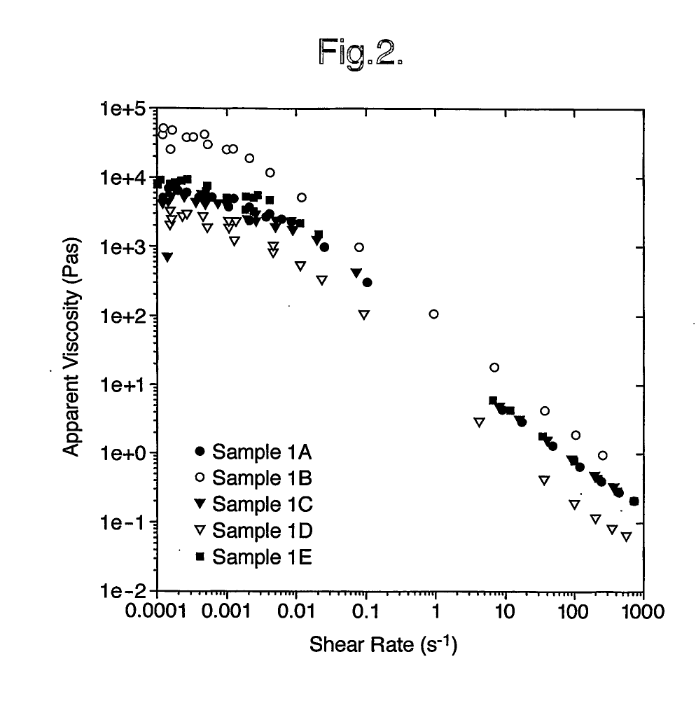

FIG. 2 shows the Theological flow curves for skin cream samples 1A to 1E as a function of shear rate.

The viscosity is measured as a function of shear rate or shear stress by those skilled in the art using the appropriate experimental configuration. The apparent yield stress is characterized as the minimum stress required to cause appreciable flow of the sample. Below the apparent yield stress the material can be regarded as either a solid or a very high viscosity fluid, while at stresses at or above the yield stress the material fractures or flows at a much reduced viscosity. For materials with an apparent yield stress above about 10 Pa, the vane geometry is most suitable because it minimises breakdown of the sample structure when the sample is loaded and minimises slippage between the material and the geometry surfaces. For yield stresses below 10 Pa, a profiled or roughened cylinder is preferred to minimise slippage. A parallel plate or cone-and-plate configurations may also be used provided extreme care is made when loading the sample to not damage the sample microstructure and that the geometry surfaces are roughened to avoid slippage. The preferred test is using a controlled stress rheometer and that the yield stress and viscosity profile is determined via a shear stress sweep with a measurement time per stress of less than 2 minutes.

The Theological properties in example set 1 were determined using a Rheometrics DSR rheometer with 50 mm parallel plates at a separation of 1 mm at 25° C. The plates were roughened by sticking P280 grade emery paper onto them. An appropriate amount of sample is placed onto the bottom platen. The top platen is quickly lowered to the tip of the sample, and then lowered very slowly and carefully at a rate less than 10 microns per second to a gap of 1 mm. Excess sample was gently cleared with a spatula. The samples were left for 5 minutes before measuring. A shear stress sweep was conducted from 0.1 Pa with 10 measurements taken per decade increase in stress. Measurements were conducted in steady state mode with a maximum duration set to 10 s. The resulted flow curve is shown in FIG. 1. The apparent yield stress is specified here as the critical point for shear thinning and is determined by intercept between the low shear region plateau and the intermediate shear region with the abrupt decrease in viscosity. These lines are indicated in FIG. 1.

Alternatively, common Theological models can be used such as the Cross model and Herschel-Buckley models to predict the yield stress based on the data and these should give a similar value for the apparent yield stress. The measurements specified are typically in error of less than 20%.

The apparent viscosity is shown as a function of shear rate in FIG. 2 for the samples 1A-1E. Values for the apparent viscosity for specified shear rates are interpolated from the data in FIG. 2.

The Theological properties in example set 2 were determined using a Paar Physica UDS200 rheometer with a serrated Z3DIN cylindrical cup of 27 mm diameter and a four pronged vane tool have the dimensions of 12 mm diameter and height of 25 mm. A shear stress sweep was conducted from 0.1 Pa with 10 measurements taken per decade increase in stress with each stress held for 5 seconds. The yield stress was evaluated in the same way as example set 2 as shown in FIG. 1.

### Example 1

Formation of a Range of Products From a Single Base Concentrate

An aqueous concentrate composition was prepared by mixing 13.5 wt % of surfactants (see table 1) with water followed by adding an aqueous composition of triethanolamide in water and heating the mixture to 80° C. The concentrate was prepared by mixing with an anchor propellor at a rotation rate of around 50 rpm in a 1 litre jacketed beaker for 10 minutes. The concentrate was cooled to 25° C. at 1° C./min under quiescent conditions; i.e. without mixing. Subsequently the concentrate was diluted with water to a concentration of 5% surfactant on total product to a volume of 100 mL by manually mixing with a spatula until a homogeneous mixture was obtained. Additional ingredients were added by mixing in with a spatula. A range of products with a variety of sensory and functional properties were produced through addition of various ingredients which are listed in Table 2. Further ingredients that were added are from: mineral oil (IPM oil), silicon oil (200 cSt), Glycerol, Simugel™,

Sunflower seed oil. The Theological properties of the lotions and creams are listed in table 3 with a general comment on their sensorial properties.

TABLE 1
Proportion of surfactant and base ingredients used in concentrate
relative to the total amount of surfactants.

| | | | Weight fraction |
|---|---|---|---|
| | Type of | | relative to |
| Ingredient | Ingredient | Trade Name | total surfactants |
| Stearic acid | Surfactant | Pristerine | 0.494 |
| Glycol Stearate | Surfactant | Ritasynt IP | 0.292 |
| Glyceryl | Surfactant | Cutina GMS-V | 0.136 |
| Monostearate | | | |
| Cetyl Alcohol | Surfactant | Lorol C16 | 0.078 |
| Triethanolamine | Organic base | TEA | 0.156 |

TABLE 2
Formulation of a range of products produced
from a 13.5% surfactant concentrate;

| | % of total mass | | | | | | |
|---|---|---|---|---|---|---|---|
| Sam- | IPM | Silicon | Glyc- | Simu- | Sunflower | Concen- | |
| ple | oil | oil | erol | gel | oil | trate | Water |
| 1A | 3 | 3 | 3 | 0 | 0 | 7.5 | 83.6 |
| 1B | 3 | 1 | 3 | 3 | 0 | 7.5 | 82.6 |
| 1C | 3 | 1 | 6 | 0 | 0 | 7.5 | 83.6 |
| 1D | 3 | 1 | 3 | 0 | 0 | 5 | 88 |
| 1E | 0 | 1 | 3 | 0 | 3 | 7.5 | 85.6 |

TABLE 3
Evaluation of properties of samples 1A to 1E and
general comments on their sensorial properties.

| | Yield | Viscosity | | |
|---|---|---|---|---|
| | Stress | (Pas) | | |
| | (Pa) | 1 s⁻¹ | 10 s⁻¹ | Sensory comments |
| 1A | 30 | 40 | 5 | fast rub in, smooth and lubricious |
| | | | | during rub-in, non-sticky, slight |
| | | | | draggy afterfeel, fast absorbing. |
| 1B | 50 | 100 | 15 | Slow rub in, sticky, silky and smooth, |
| | | | | thick lotion, smooth |
| 1C | 30 | 40 | 5 | Slow rub in, slightly greasy, low |
| | | | | tackiness, slight dragginess, extra |
| | | | | moisturing. |
| 1D | 8 | 12 | 1.5 | fast absorption, nonsticky, draggy, |
| | | | | thin lotion, fast spreading |
| 1E | 30 | 40 | 5 | Slight tackiness, very draggy |
| | | | | afterfeel, oily, smooth rub-in feel |

### Example 2

Formation and Dilution of a Range of Concentrated Bases

Sample F

The procedure in example 1 was followed for the production of a concentrate at 15% surfactant concentration. This was diluted with water alone under ambient conditions to a final surfactant concentration of 5% and 10%.

Rheological Properties:

5% surfactants: Yield stress=65 Pa

10% surfactants: Yield stress=160 Pa

Sample G

The procedure in example 1 was followed for the production of a concentrate at 15% surfactant concentration except that shear was applied throughout the cooling process using an anchor impeller at a rotation rate of 50 rpm. This was diluted with water alone under ambient conditions to a final concentration of 5 % and 10 %.

Rheological Properties:

5% surfactants: Yield stress=1 Pa

10% surfactants: Yield stress=30 Pa

The obtained products were softer and less textured than those obtained under quiescent cooling of the aqueous concentrate (see sample F).

Sample H

The procedure in example 1 was followed for the production of a concentrate except that a concentrate consisting of 30% surfactants was made and triethanolamine was mixed in with the surfactants prior to the addition of the aqueous phase. This was diluted with water alone under ambient conditions to a final concentration of 5% and 10%.

Rheological Properties:

5% surfactants: Yield stress=30 Pa

10% surfactants: Yield stress=125 Pa

1. Process for the preparation of a personal care composition, which process comprises the steps of
a) providing an aqueous concentrate composition comprising a neutralising base and 6 to 90 wt % of a surfactant at a temperature of from 60 to 150° C., said surfactant comprising fatty acid,
b) cooling the composition obtained in step (a) to a temperature below 60° C., preferably to a temperature from 5 to 30° C., more preferred from 10 to 25° C.
c) diluting of the base composition until a surfactant concentration between 1 and 30% is obtained
d) setting the sensory perception and the functionality of the composition by adding at least one ingredient selected from the group comprising sensory ingredients, functional ingredients or a combination thereof,
e) Optionally adding promotionals and/or emotionals or other minor ingredients;
wherein steps c, d, and e may be performed simultaneously or sequentially in any order.

2. Process according to claim 1 wherein the surfactant comprises fatty acid and wherein the amount of neutralising base in step (a) is such that the fatty acid is partially neutralised.

3. Process according to claim 1 wherein the aqueous concentrate composition in step (a) comprises a fatty acid and at least one component selected from the group comprising long chain fatty alcohol, fatty acid esters, glycerol monostearate, cetylalcohol, polyethylene glycol or a combination thereof.

4. Process according to claim 1 wherein in step (b) below a temperature of 65° C., the cooling is carried out quiescently.

5. Process according to claim 1 wherein the neutralising base in the concentrate is selected from the group comprising organic base, such as triethanolamine, diethanolamine, monoethanolamide, triisopropanalamine, inorganic base such as KOH, or a mixture of inorganic and organic base, and/or a non ionic surfactant.

6. Process according to claim 1 wherein the personal care composition is a cream or a lotion.

7. Process according to claim 1 wherein in step (d) functional and sensorial properties of the composition are set by inclusion of ingredients preferably selected from the group comprising oils, polymers, and glycerol or a combination thereof.

8. Process according to claim 1 wherein in step (c,d,e) the temperature is from 5 to 90° C.

9. Process according to claim 1 wherein the dilution in step (c) is with an aqueous composition.

10. Process according to claim 1 wherein the temperature of the aqueous concentrate composition of step (a) is from 70 to 90° C.
